(19) United States
(12) Patent Application Publication
(10) Pub. No.: US 2006/0129243 A1
(43) Pub. Date: Jun. 15, 2006

(54) Interbody spinal device

(76) Inventors: Hee K. Wong, Singapore (SG); James Goh, Singapore (SG)

Correspondence Address: DICKSTEIN SHAPIRO MORIN & OSHINSKY LLP, 2101 L Street, NW, Washington, DC, 20037, US

(21) Appl. No.: 11/230,548

(22) Filed: Sep. 21, 2005

Related U.S. Application Data
(60) Provisional application No. 60/611,603, filed on Sep. 21, 2004.

(51) Int. Cl. A61F 2/44

(52) U.S. Cl. 623/17.16

(57) ABSTRACT

An interbody spinal device for insertion into an intervertebral disc space of a vertebrate animal, where the device is adapted to rotate within the intervertebral disc space upon insertion. The invention also provides a method of distracting and/or maintaining two adjacent vertebrae of a vertebrate animal until the two adjoining vertebrae are fused, the method comprising: (a) creating an intervertebral disc space between the two adjacent vertebrae through an aperture; and (b) inserting an interbody spinal device through an aperture into the intervertebral disc space.

This application claims the benefit of U.S. Provisional Application No. 60/611,603, filed Sep. 21, 2004.

### FIELD OF THE INVENTION

The present invention relates to the field of orthopaedic surgery. In particular, the present invention relates to interbody spinal devices.

### BACKGROUND OF THE INVENTION

Surgical intervention in the treatment of degenerative diseases of the spine is often in the form of an interbody spinal fusion performed at the diseased level. In performing an interbody fusion, the space created by the removal of the intervertebral disc has to be supported and maintained in the correct anatomical position for a suitable length of time so that new bone growth can occur between the adjacent vertebrae. This new bone growth immobilizes the diseased spinal level, thus eliminating the back pain that patients complain of. Complete lack of motion at the implant-vertebral body interface has been shown to be a critical condition for achieving solid fusion and a successful clinical outcome.

Interbody spinal fusion implant devices that are currently used in clinical practice are either cylindrical or rectangular and are made of biocompatible materials like titanium, titanium alloy and medical grade stainless steel. An example of current devices is that taught by U.S. Pat. No. 5,607,424. Using devices of the current art, the cortical endplates of the adjacent vertebral bodies have to be removed to seat the implants properly. This causes two problems. The first one is the added time of surgery and blood loss. The second is that the weaker cancellous bone is exposed and the implant is placed on this bed of weak bone. This has been known to cause implant subsidence into the weaker cancellous bone bed. This results in a loss of distraction and in most cases, the formation of a pseudarthrosis. Even with careful preparation of the intervertebral disc space (otherwise denoted simply as the disc space herein under), maximal contact at the implant-bone interface is difficult to achieve due with the currently used implants. This compromises the initial stability of the construct and it has been shown that initial stability is a critical factor in determining the final outcome of the surgery.

Initial stability of the implanted spine may be dependent on the size of the implants used, with larger implants giving better initial stability. A problem that has been shown to exist when using the currently available interbody spinal fusion implant devices in an Asian population is the near total facetectomy needed to insert implants large enough to provide the initial stability required. The loss of the facet joints seriously compromise the rotational stability of the implanted spine. In current practice, to facilitate osteoinduction, the intervertebral disc space is packed with bone graft. Due to the inherent risk of disease transfer and the possibility of rejection of donor bone, autogenous bone, harvested intra-operatively, is used. This adds to surgical time, increases blood loss and risk of infections, and is a source of postoperative pain to patients.

Accordingly, there is a need in this field for improved spinal implant devices as well as of surgery techniques to minimize trauma and to facilitate the patient's recovery.

### SUMMARY OF THE INVENTION

The present invention addresses the problems above, and provides a new and useful interbody spinal device.

In particular, the device according to the invention is suitable for use in interbody spinal fusion of two adjacent vertebrae.

According to a first aspect, the present invention provides a device for insertion into an intervertebral disc space of a vertebrate animal. In particular, there is provided an interbody spinal device for insertion into an intervertebral disc space of an animal, wherein the device is adapted for rotational insertion into an intervertebral disc space upon insertion. More in particular, there is provided a method for distracting and/or maintaining two adjacent vertebrae of an animal.

The device according to the invention has a shape substantially arcuate. It may comprise at least a convex side surface and at least another side surface, the convex and another side surfaces being opposite one to the other. The another side surface may be concave.

More in particular, the device comprises:

a leading edge;

a back end being opposite to the leading edge;

a first surface;

a second surface being opposite to the first surface;

a convex side surface; and

a concave side surface being opposite to the convex side surface.

In particular, the first and second surfaces of the device may be convex.

The device may be made of a biodegradable and/or biocompatible material, for example, poly-caprolactone (PCL). The device may be made of porous material. For example, the device may be made of or comprises a material that has an open cell structure.

The device according to the invention may further comprise growth factors and/or stem cells, for example, mesenchymal stem cells.

The device may also be made of a non-radio-opaque material.

According to another aspect, the present invention provides a method of inserting an interbody spinal into the spine of an animal, the method comprising:
(a) creating an intervertebral disc space between the two adjacent vertebrae through an aperture; and
(b) rotationally inserting an interbody spinal device through an aperture into the intervertebral disc space.

The device inserted in step (b) is an interbody spinal device which is adapted to rotate within the intervertebral disc space upon insertion. In particular, the method is a method of distracting and/or maintaining two adjacent vertebrae of a vertebrate animal.

The device according to any aspect of the invention can be inserted from a unilateral posterior side of the spine into the intervertebral disc space. In particular, the device according to any aspect of the invention may comprise at least one convex side surface and at least another side surface, the convex and another side surfaces being opposite one to the other. In particular, the another side surface may be concave. Upon insertion of the device into the intervertebral disc, the convex side surface contacts the lateral and anterior portion of the annulus fibrosus of the disc and the device rotates with the application of a force in the anterior-posterior direction. In particular, upon insertion of the device into the intervertebral disc, the convex side surface fits with the lateral and anterior portion of the annulus fibrosus of the disc.

The animal is a vertebrate. It may be a mammal, for example, a human being or a non-human mammal.

There is also provided a method for the manufacture of a device according to any aspect of the invention by using biodegradable and/or biocompatible material. For example, the material is poly-caprolactone (PLC).

### BRIEF DESCRIPTION OF THE DRAWINGS

FIG. 1 shows one example for the shape of the device of the present invention designed for unilateral posterior insertion. The first or upper surface (a) and the second or lower surface (c) are contoured (convex) to match the contours of the cortical endplates. The device is inserted with the leading edge (f) entering the disc space first. As the device is inserted, the convex side surface (b) contacts the lateral and anterior portion of the annulus fibrosus and due to its curved or arcuate shape, turns in response to a force in the anterior-posterior (AP) direction. The side surface (d) is concave and it is opposite to the side convex surface (b). The back end (e) is opposite to the leading edge (f).

FIG. 2 shows the different views of the device of FIG. 1.

FIG. 3 shows another example for the shape of the device of the present invention also designed to for unilateral posterior insertion but with a more tapered shape than the example of FIG. 1. The first or upper surface (a) and the second or lower surface (c) are contoured to match the contours of the vertebral endplates. The edge (f) is the leading edge during insertion of the device.

FIG. 4 shows the different views of the device of FIG. 3.

FIGS. 5 and 6 are cross-sectional plan views showing the process of insertion of the two examples of the device of the present invention each into an intervertebral disc space. FIGS. 5A and 6A show the device being introduced through the aperture created in the annulus fibrosus of the intervertebral disc. FIGS. 5B and 6B show the convex side surface of the device contacting the lateral and anterior portion of the annulus fibrosus and rotating within the intervertebral disc space. FIGS. 5C and 6C show the device in its final position within the intervertebral disc space.

### DETAILED DESCRIPTION OF THE INVENTION

The present invention provides an interbody spinal device for insertion into the spine of a vertebrate animal. In particular, an interbody spinal device for insertion into an intervertebral disc space of a vertebrate animal. More in particular, device according to the invention is suitable for insertion from a unilateral posterior approach into the intervertebral disc space when performing lumbar interbody fusion.

The device may also be referred interchangeably as an interbody spinal device or interspinal device and the terms “device”, “implant” or “implant device” are used synonymously.

The present invention is designed to overcome the shortcomings of the implants currently used for spinal fusion.

According to a first aspect, the present invention provides an interbody spinal device for insertion into an intervertebral disc space of a vertebrate animal, wherein the device is adapted to rotate within the intervertebral disc space upon insertion. In particular, the invention provides a contoured interbody spinal device. More in particular, there is provided a method for distracting and/or maintaining two adjacent vertebrae of an animal. The adjacent two vertebrae are maintained due to the unique geometrical design of the device.

More particularly, there is provided a device which has a shape substantially arcuate.

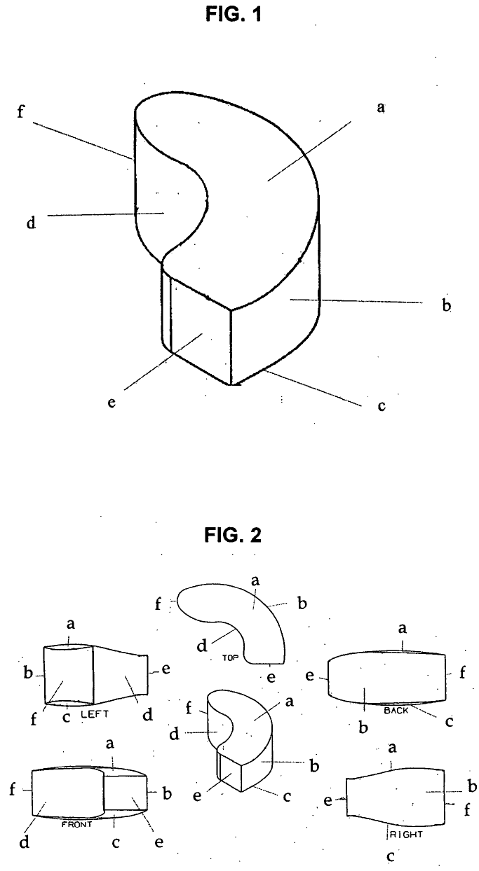

With reference to FIGS. 1 and 6 of an embodiment of the invention, the invention will be described in relation to its position to a standing human patient, just before the invention is inserted into the intervertebral disc space. All references to orientational directions such as “upper” and “lower” in reference to the surfaces of the device as well as “lateral” and “medial”, “anterior” and “posterior” in relation to structures of the patient's body, are in relation to the standing human patient and as commonly understood in the field of surgery.

The invention has a substantially arcuate shape, that is, its long axis has a visible degree of curvature. The device may comprise several major surfaces: a first or upper surface (a), a second or lower surface opposite (c) the first or upper surface; a convex side surface (b) and another side surface (d) opposite the convex surface. The another side surface may be concave. The invention may also comprise two portions: a front or leading edge (f) and a back or trailing end (e) opposite the front or leading edge. The edges between the major surfaces may be further bevelled.

More in particular, the device comprises:

a leading edge;

a back end being opposite to the leading edge;

a first surface;

a second surface being opposite to the first surface;

a convex side surface; and

a concave side surface being opposite to the convex side surface.

The upper (first) and lower (second) surfaces of the device may be of any shape. For example, the upper and lower surfaces may be contoured (convex) to match the contours of the adjacent vertebral endplates. This removes the need to cut the cortical endplates of the vertebrae and with it the whole host of problems which may accompany the removal of the cortical endplate causes.

By using contoured upper and lower surfaces, maximal contact for stability at the implant-vertebral body interface is ensured. This will eliminate instability and micro motion at the interface, which have been shown to increase the chances of failure of the surgery.

The substantially arcuate shape of the implant allows the implant to be inserted with the minimal removal of bone such as the removal of only one facet joint of a vertebra. This translates to shorter operating time and better initial stability of the construct.

The device is shaped in such a way that it facilitates smooth and easy insertion into the disc space from a unilateral posterior approach. The outer surface of the device, is curved in such a way as to mimic the curvature of the inside edge of annulus fibrosus of the disc.

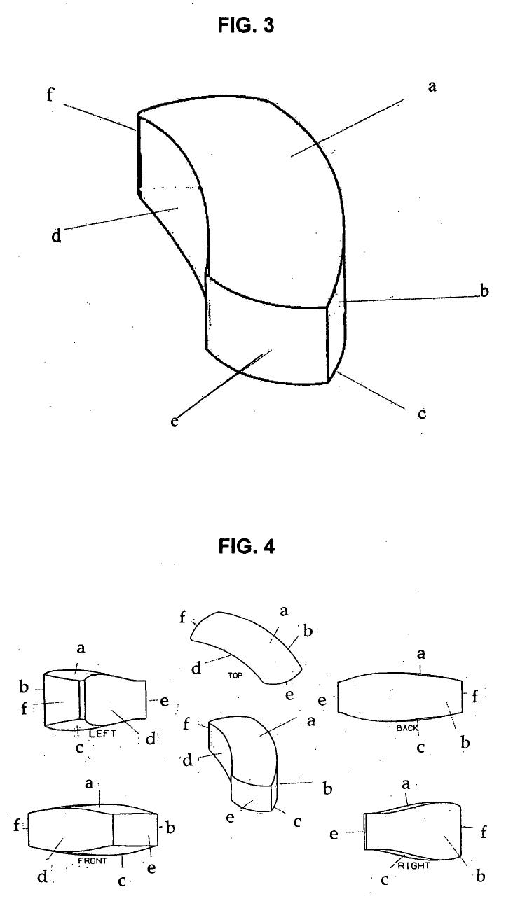

While the shape of the implant is generally arcuate, several parameters relating to the shape of the device may be varied within the scope of the present invention. The degree of curvature of its arcuate shape, the profile of the leading edge, and the number and size of bevels between the major surfaces may be varied. The width of the implant (that is, the distance between the convex and another side surfaces) may be constant or decreasing from the back end to the leading edge, making the device tapered along its length such as that shown in FIG. 3.

The device may also be sized to suit different patients of different age and race. The size may be varied by varying its length, width or height (thickness between the upper and lower surfaces). It may be of larger dimensions for Caucasian patients or smaller to make it suitable for use in Asian patients.

To overcome the problems associated with the use of autogenous bone grafts for osteoinduction, this device may have a porous or open cell structure, in one embodiment, to act as a carrier for bone morphogenetic proteins and as a scaffold for mesenchymal stem cells. Bone morphogenetic proteins have been shown to give superior results in spinal fusion as compared to autografts. Also, the whole host of problems associated with intra-operative harvesting of bone graft is eliminated with the use of bone morphogenetic proteins. The device will be able to act as a carrier for mesenchymal stem cells and appropriate growth factors capable of osteoinduction (such as bone growth factor or BMP and transforming growth factor beta or TGF-β). The material used for the device may further comprise such growth factors or stem cells by being coated or, impregnated with such growth factors or stem cells, according to standard techniques known to the skilled person.

The device may be made out of biocompatible, biodegradable material in another embodiment. For example, it may be made of biocompatible, biodegradable polymer material. In particular, the polymer material is polycaprolactone (PCL). The use of this material obviates the problems associated long-term presence of a foreign body in the human body. The structure of the device may be porous. For example, the device may be made in such a way there are interconnecting pores throughout the structure like a sponge and open to the surroundings at the surfaces of the implant. Accordingly, there is also provided a device made of a material that has an open cell structure.

The device may also be made of a non-radio-opaque (that is, radiolucent) material, or a material that is transparent to x-rays as well as other diagnostic imaging energies and wavelengths. This obviates the problems associated with metal implants with regards to stress shielding, long-term presence of a foreign body in the human body and problems with radiological assessment of fusion progress due to the radio-opaque nature of metals.

According to another aspect, the present invention provides a method of inserting an interbody spinal into the spine of an animal, the method comprising:
(a) creating an intervertebral disc space between the two adjacent vertebrae through an aperture; and
(b) rotationally inserting an interbody spinal device through an aperture into the intervertebral disc space.

In particular, the device inserted in step (b) is an interbody spinal device which is adapted to rotate within the intervertebral disc space upon insertion. In particular, the method is a method of distracting and/or maintaining two adjacent vertebrae of a vertebrate animal. More in particular, until the two adjoining vertebrae are fused.

The device according to any aspect of the invention may be inserted from a unilateral posterior side of the spine into the intervertebral disc space. In particular, the device according to any aspect of the invention has a shape that is substantially arcuate. More in particular, the device may comprise at least one convex side surface and at least another side surface, the convex and another side surfaces being opposite one to the other. In particular, the another side surface may be concave. Upon insertion of the device into the intervertebral disc, the convex side surface contacts the lateral and anterior portion of the annulus fibrosus of the disc and the device rotates with the application of a force in the anterior-posterior direction. In particular, upon insertion of the device into the intervertebral disc, the convex side surface fits with the lateral and anterior portion of the annulus fibrosus of the disc.

In particular, the leading edge is lower in height thus allowing easy entry through an aperture into the intervertebral disc space. As the device is inserted further, due to the increase in height at the domed region, it distracts the two adjacent vertebrae. The device is then manoeuvred around in the disc space until the leading edge is at the far side of the disc. The trailing edge also has a lower height. The convex side is higher (thicker) than the concave side. Following the insertion of the device, the convex side will lie anterior and due to its higher height than the concave side, which is now posterior, the lordortic curve is achieved and maintained.

The vertebrate may in particular be a mammal, for example, a human being or a non-human mammal.

According to another aspect, there is also provided a method for the manufacture of a device according to any aspect of the invention by using biodegradable and/or biocompatible material. For example, the material is polycaprolactone (PLC).

Having now generally described the invention, the same will be more readily understood in the following examples, with reference to the figures, which are provided by way of illustration, and are not intended to be limiting of the present invention.

### EXAMPLES

### Example 1

### Fabrication of the Implant

The implant may be fabricated by computer-aided design and computer-aided manufacturing methods of a suitable biocompatible, biodegradable polymer such as PCL. A block of PCL may be reduced to the desired shape and size by suitable machining methods and then sterilized by known methods for implantation. Alternatively, the implant may be cast or injection-moulded or formed by other methods known in the art for polymers. A person skilled in the art of polymer science will appreciate that many alternatives may be used to fabricate the device of the present invention to possess desire levels of density, porosity or rates of biodegradation.

The surgeon implanting the device may choose from a variety of sizes and shapes. Alternatively, one or more precision implants may be custom-made for each patient based on diagnostic images obtained prior to the surgery. The implants may then be further treated to encourage new bone growth (osteoinduction) by either impregnation with suitable growth factors or mesenchymal stem cells, or coated with an osteoinductive or osteoconductive coating such as hydroxyapatite.

### Example 2

### Implantation of the Device

A person skilled in the art in the field of orthopaedic surgery, particularly one specializing in the spine, will appreciate that many variations may be made in the procedure to implant the device of the present invention without departing from the scope of the present invention.

The surgery is performed under general anaesthesia. The patient is positioned prone on the operating table. Care is taken to pad bony or exposed areas to avoid under pressure on the soft tissues and neurovascular bundles. There should be no compression on the abdomen to reduce epidural vein congestion. The operative field is cleaned with a suitable disinfectant, and then draped.

The interbody spinal device is designed for insertion through a posterior or postero-lateral surgical approach to the spine; and thence via a unilateral trans-facetal approach to the disc, although its shape and dimensions will allow its insertion through the anterior, antero-lateral, or lateral approach to the spine and the intervertebral disc.

The approach to the spine and the intervertebral space is made either through a single midline incision, or a paraspinal “Wiltse” incision. The muscles are retracted and the approach brought down to the lamina and facet joints of the lumbar segment to be fused. On the side chosen for insertion of the device, partial or subtotal facetectomy is performed. The underlying ligamentum flavum that overlies the intervertebral foramen is defined and is excised, exposing the lateral aspect of the spinal canal and the intervertebral foramen. The dural sac and the segmental traversing nerve root is gently retracted medially, while the exiting nerve root is identified in the upper region of the foramen and protected. Epidural bleeding is controlled by bipolar cautery. The intervertebral disc is exposed between the traversing and exiting nerve root at the lateral aspect of the spinal canal and in the intervertebral foramen. The annulus fibrosus of the intervertebral disc is then incised to create an aperture through which the disc is entered; and internal contents of the disc removed. The adjacent vertebrae are distracted to enable optimal clearance of the intervertebral disc. The cartilaginous end-plate is separated from the bony end-plate of the adjacent vertebral bodies; end-plate preparation is completed using curettes, exposing but not cutting into the bleeding bone surfaces of the end-plates.

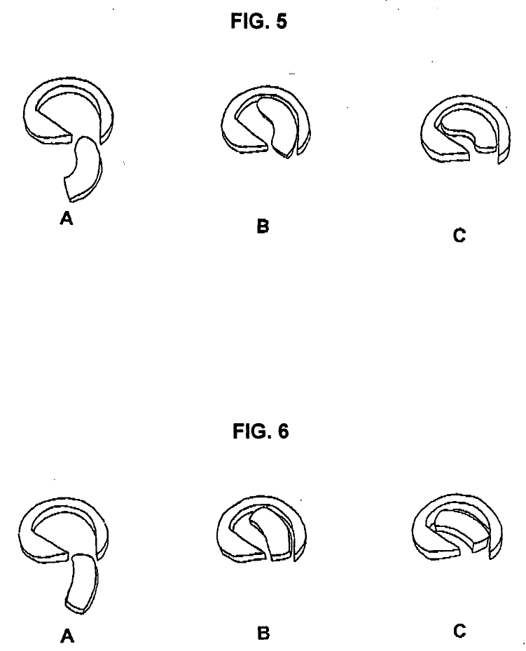

The aperture into the disc may be enlarged slightly where necessary to facilitate initial entry placement of the contoured device of the present invention. The interbody spinal device is inserted on its long axis; gradually turning towards the opposite side with progressive insertion as shown in FIGS. 5A-C and 6A-C. Care is taken to turn the device to the opposite side after inserting it in order to avoid anterior penetration of the anterior annulus fibrosus. Care is also taken to ensure that the device has entered the disc space before turning to avoid device intrusion into the spinal canal. It is also important to achieve adequate clearance of the disc space to the extent as shown in FIGS. 9A-C and 10A-C so that the entire device can be fitted into the disc space. The device should be correctly sized in height to achieve distraction of the intervertebral space as well as a snug fit, and to avoid extrusion of the device. Pedicle screw fixation of the spinal segment is necessary to complete the stabilization procedure. The device should preferably be used with supplementary posterior fixation. Thereafter, closed suction drains are inserted, and the surgical wound is closed in the standard manner.

A person skilled in the field of orthopaedic surgery, will appreciate that the shape of the device as defined by the major surfaces, allow the device to be inserted in an anterior-posterior (AP) direction into the disc space from the unilateral posterior approach described above.

### Example 3

### Selected Biomechanical Test Results

Biomechanical tests were performed on 10 cadaveric specimens. The specimens were tested in three configurations, (1) intact; (2) following removal of intervertebral disc (before implant); and (3) following implantation of the device of the present invention. The stiffness values of the specimens were calculated and the data were normalised to that of the intact specimens. The results are as shown in the Table 1 below:

TABLE 1

| | Lateral Bending | Flexion | Extension | Axial Rotation |
|---|---|---|---|---|
| Before Implant | 80% | 71% | 55% | 78.5% |
| After Implant | 87.5% | 92% | 67% | 86% |

The results showed that following removal of the intervertebral discs, the stiffness of the specimens in the four modes of testing, ie lateral bending, flexion, extension and axial rotation were reduced. After implantation of the device of the present invention, the stiffness of the specimens improved as shown in the above Table 1.

### Example 4

### An Embodiment of the Device

An embodiment of the device of the present invention incorporates the features as listed above and according to the FIGS. 1 to 4. The device of a first embodiment is made out of porous PCL and is coated or impregnated with one or more growth factors and one or more types of mesenchymal stem cells. The shape of the device allows it to be readily inserted from a unilateral posterior approach into the disc space, through a small aperture. Its convex contoured upper and lower surfaces obviate the need to cut the endplates of adjoining vertebrae and prevent movement between these vertebrae.

The growth factors transform the stem cells and speed up osteoinduction even as the device biodegrades over time. Progress may be readily tracked by any suitable diagnostic imaging technique such as x-ray as the device is radiolucent. When the two vertebrae are fused, there is no remnant of the device left.

While the present invention is described for use in human patients, the invention can also be readily applied to other vertebrate animals such as mammals or birds in the field of veterinary surgery.

What is claimed is:

1. An interbody spinal device for distracting and/or maintaining two adjacent vertebrae of an animal, wherein the device is adapted for rotational insertion into an intervertebral disc space.

2. The device according to claim 1, wherein the device has a shape that is substantially arcuate.

3. The device according to claim 1, comprising at least a convex side surface and at least another side surface, and wherein the convex surface and said another side surface are opposed to each other.

4. The device according to claim 3, wherein the another side surface is concave.

5. The device according to claim 1, the device comprising:
a leading edge;
a back end being opposite to the leading edge;
a first surface;
a second surface being opposite to said first surface;
a convex side surface; and
a concave side surface being opposite to said convex side surface.

6. The device according to claim 5, wherein the first and second surfaces are convex.

7. The device according to claim 1, wherein the device is made of a biodegradable and/or biocompatible material.

8. The device according to claim 1, wherein the device is made of poly-caprolactone (PCL).

9. The device according to claim 1, wherein the device comprises material which has an open cell structure.

10. The device according to claim 1, wherein the device further comprises growth factors and/or stem cells.

11. The device according to claim 1, wherein the device is made of a non-radio-opaque material.

12. A method of distracting and/or maintaining two adjacent vertebrae of an animal, the method comprising:
(a) creating an intervertebral disc space between the two adjacent vertebrae through an aperture; and
(b) rotatationally inserting an interbody spinal device through the aperture into the intervertebral disc space.

13. The method according to claim 12, wherein the device is inserted from a unilateral posterior side of the spine into the disc space.

14. The method according to claim 12, wherein the device has a shape that is substantially arcuate.

15. The method according to claim 12, wherein the device comprises at least one convex side surface and at least one concave side surface, wherein the convex surface and said concave side surfaces are opposed to each other, and wherein upon insertion of the device into the intervertebral disc space, the convex side surface fits with the lateral and anterior portion of the annulus fibrosus of the disc.

16. The method according to claim 12, the device comprising:
a leading edge;
a back end being opposite to the leading edge;
a first surface;
a second surface being opposite to the first surface;
a convex side surface; and
a concave side surface being opposite to the convex side surface.

17. The method according to claim 16, wherein the first and second surfaces are convex.

18. The method according to claim 12, wherein the device is made of a biodegradeble and/or a biocompatible material.

19. The method according to claim 12, wherein the device further comprises growth factors and/or stem cells.

20. The method according to claim 12, wherein the device is made of a non-radio-opaque material.

21. The method according to claim 12, wherein the animal is a human being.
